(19) United States
(12) Patent Application Publication
(10) Pub. No.: US 2023/0124764 A1
(43) Pub. Date: Apr. 20, 2023

(54) LACTOBACILLUS HARBINENSIS AND APPLICATION THEREOF

(71) Applicant: South China University of Technology, Guangzhou, Guangdong (CN)

(72) Inventors: Li LI, Guangdong (CN); Yin ZHENG, Guangdong (CN); Yongtao FEI, Guangdong (CN)

(21) Appl. No.: 17/287,506

(22) PCT Filed: Oct. 18, 2019
(86) PCT No.: PCT/CN2019/112046

§ 371 Date: Jul. 7, 2021

(30) Foreign Application Priority Data
Oct. 22, 2018 (CN) 201811228606.5

(51) Int. Cl. A23C 11/10; C12N 1/20

(52) U.S. Cl. CPC A23C 11/106; C12N 1/205; C12R 2001/225

(57) ABSTRACT

The invention discloses a Schleiferilactobacillus harbinensis (former name Lactobacillus harbinensis) M1 and use thereof. The preservation number of Schleiferilactobacillus harbinensis M1 is GDMCC No. 60305. The strain is sensitive to antibiotics such as ampicillin, tetracycline and chloramphenicol, and its safety meets the requirements of EFSA. Its supernatant has a significant inhibitory effect on Listeria monocytogenes, Streptococcus hemolyticus and Staphylococcus aureus. The present invention also protects the use of Schleiferilactobacillus harbinensis M1 in fermented soymilk; using the bacteria to ferment soymilk alone has the advantages of strong acid production ability, high amount of viable bacteria, few or even completely no beany flavor substances, and significant higher content of characteristic aroma components of fermented milk, which greatly improves the sensory flavor of fermented soymilk.

### CROSS-REFERENCE TO RELATED APPLICATIONS

This application is the U.S. national stage of International Patent Application No. PCT/CN2019/112046, filed on Oct. 18, 2019 and entitled LACTOBACILLUS HARBINENSIS AND APPLICATION THEREOF, which claims the benefit of priority under 35 U.S.C. § 119 from Chinese Patent Application No. 201811228606.5, filed Oct. 22, 2018. The disclosures of the foregoing applications are incorporated herein by reference in their entirety.

### SEQUENCE LISTING

The entire content of a Sequence Listing titled “Sequence_Listing.txt,” created on Sep. 9, 2021 and having a size of 3 kilobytes, which has been submitted in electronic form in connection with the present application is hereby incorporated by reference herein in its entirety.

### TECHNICAL FIELD

The present invention relates to the field of probiotics, in particular to Schleiferilactobacillus harbinensis M1 and use thereof. The Schleiferilactobacillus harbinensis M1 can be used as a probiotic and as a starter in fermented soy milk.

### BACKGROUND

Probiotics are live microorganisms that are beneficial on the health of the host when a certain amount (usually above 10⁶ CFU/g) of them is provided. Only those microorganisms that can tolerate gastrointestinal digestion, that adhere to and colonize on the epithelial cells of the small intestine, and that inhibit the growth of pathogenic microorganisms can provide beneficial effects to the health of the host. Commonly used probiotics mainly include lactobacilli, bifidobacteria, and gram-positive cocci, such as Streptococcus thermophilus. Their probiotic functions mainly include enhancing immunity, regulating intestinal flora and improving gastrointestinal function, anti-oxidation and anti-aging, reduction of cholesterol and improving blood lipids.

Soybeans are rich in high-quality protein, unsaturated fatty acids, oligosaccharides, isoflavones and saponins and other functional ingredients. They are environmentally friendly, healthy and economical, and are traditional important plant protein resources in China having important economic value and social value. The soy protein beverage prepared from fermenting soybeans with probiotics contains a large number of live probiotics, transforms isoflavone to easily absorbed aglycon through biotransformation, and degrades soy protein to polypeptides and amino acids, at the same time producing functional factors like γ-aminobutyric acid and vitamin B. Therefore, the probiotic fermented soymilk is receiving wide attention from food scientists and modern consumers. However, the product still has some sensory quality defects. For example, compared with fermented cow milk, probiotic fermented soymilk is significantly weak in taste and flavor, wherein the beany flavor is so prominent that it seriously affects the product's acceptability and restricts the product's industrial application.

The beany flavor of soybean food mainly comes from volatile fat oxidation degradation products such as n-hexanal, nonanal, 1-octene-3-ol, etc. formed by the catalysis of lipoxygenase. At present, treatments to reduce the unfavorable flavor mainly include enzyme inactivation, oxygen isolation, and biotransformation. Among them, the effect of fermenting with probiotics to reduce the beany flavor is closely related to the species of the probiotics. Since most kinds of the lactic acid bacteria currently used in commercial applications are derived from fermented dairy products, the application of these bacteria in fermented soymilk often exhibits poor adaptability, mainly including poor fermentation quality and poor product flavor.

### SUMMARY OF THE INVENTION

Regarding the problems existing in the prior art, the purpose of the present invention is to provide Schleiferilactobacillus harbinensis M1 suitable for fermented foods, especially fermented soybean foods. The strain has good safety and probiotic functions, and can effectively utilize various oligosaccharides in soybeans to significantly improve and enhance the sensory flavor of legumes.

Yellow serofluid of bean curd is the yellow drain produced in the production process of bean curd. Because the yellow serofluid is still rich in nutrients, it is very suitable for the growth and reproduction of microorganisms, leading to naturally acidification during storage. In parts of China, there is a tradition of using the acidified yellow serofluid as a coagulant to prepare bean curd. Because such kind of acidified-yellow-serofluid bean curd has a delicious taste and unique flavor, it has been favored by more and more consumers in recent years. Due to long-term domestication, the microorganisms in the yellow serofluid may effectively utilize legume components such as raffinose, stachyose and isoflavones, and produce a good flavor. The present invention screened out a strain of Schleiferilactobacillus harbinensis with excellent fermentation performance in soybean substrate on the basis of in-depth research on acidified yellow serofluid of bean curd, and the strain has potential probiotic function and important application value.

The purpose of the present invention is achieved through the following technical solutions:

Schleiferilactobacillus harbinensis M1 with an accession number of GDMCC Accession No. 60305 was deposited at 5^{th} Floor, Building 59, No. 100, Guangdong Microbial Culture Collection Center, Xianlie Middle Road, Guangzhou on Dec. 20, 2017.

The Schleiferilactobacillus harbinensis M1 may be used as probiotics.

The Schleiferilactobacillus harbinensis M1 is sensitive to antibiotics including ampicillin, vancomycin, gentamicin, kanamycin, streptomycin, erythromycin. clindamycin, tetracycline and chloramphenicol, and the safety meets the requirements of EFSA.

The supernatant of the Schleiferilactobacillus harbinensis M1 is effective on inhibitory of Listeria monocytogenes, Salmonella typhimurium, Escherichia coli 0157, Enterobacter sakazakii, Streptococcus hemolyticus and Staphylococcus aureus.

The Schleiferilactobacillus harbinensis M1 is resistant to gastric juice, intestinal juice and bile salts, and can be colonized on small intestinal epithelial cells.

The Schleiferilactobacillus harbinensis M1 may be used in fermented soybean milk.

The Schleiferilactobacillus harbinensis M1 has utilization rates of sucrose, stachyose and raffinose substantially the same as the utilization rate of glucose.

The Schleiferilactobacillus harbinensis M1 completely removes the beany flavor component n-hexanal in the soymilk.

The Schleiferilactobacillus harbinensis M1 increases the milk aroma components diacetyl and acetoin.

In the present invention, Schleiferilactobacillus harbinensis M1 is obtained by using a sterile sampling bottle to collect naturally fermented acidified yellow serofluid sample on site followed by a pour plate method. The biological activity characteristics of this strain are as follows: the colony on the MRS solid medium plate is protruding, round, generally 1-3 mm in diameter, off-white, opaque, moist and smooth; the strain are Gram-positive sporeless bacterium with a thin and short rod shape arranged in pairs or piles; the bacteria is glucose hetero-fermentative with a wide growth temperature range (grow normally at 20-45° C.) and a fast growth speed; the results of routine physiological and biochemical experiments show that the bacteria M1 is Gram-positive and catalase negative, and non-exercise. The 16S rDNA sequence was further analyzed, and through BLAST comparison, it was identified as Schleiferilactobacillus harbinensis (former name Lactobacillus harbinensis).

In the present invention, Schleiferilactobacillus harbinensis M1 has the characteristics of probiotics and shows good tolerance to simulated gastric juice and simulated intestinal juice. It can adhere to small intestinal epithelial cells Caco-2 and can effectively inhibit the reproduction of food-borne pathogenic bacteria such as Listeria monocytogenes, Salmonella typhimurium, Escherichia coli 0157, Enterobacter sakazakii, Streptococcus hemolyticus and Staphylococcus aureus. At the same time, the growth of the bacteria can effectively utilize sucrose, stachyose and raffinose, and is suitable for the production of fermented foods rich in plant oligosaccharides. The fermented soymilk prepared by the bacteria has high viable bacteria amount and acidity, and the beany flavor components in the volatile flavor components are reduced, while the milk aroma components diacetyl and acetoin are increased, which effectively enhances the sensory flavor of the product.

Compared with the existing commercial bacteria, the present invention has the following advantages and beneficial effects:

(1) The bacteria Schleiferilactobacillus harbinensis M1 of the present invention has good tolerance to the gastrointestinal tract, which can reach to the small intestine alive, adhere and colonize on epithelial cells, and has a strong inhibitory effect on a variety of food-borne pathogens. It has the potential to become dominant bacteria in the intestinal tract and can provide the host with a good probiotic effect.

(2) The strain of the present invention has a very high utilization rate of oligosaccharides such as raffinose, stachyose, and sucrose. It can grow and reproduce in soybeans and other plant materials rich in such oligosaccharides, which is a new adaptive strain for the production of plant-derived foods like fermented soybean.

(3) The stain of the present invention can reduce the content of beany flavor substances such as n-hexanal, nonanal and 1-octene-3-ol in fermented soymilk, increase aroma components such as diacetyl and acetoin, and introduce characteristic milk aroma to the fermented soymilk, which significantly improves the sensory flavor of fermented soymilk.

(4) The strain of the present invention has a wide growth temperature range, a fast growth rate, simple cultivation conditions, easy industrial production and management, and broad development and application prospects.

### BRIEF DESCRIPTION OF THE DRAWINGS

FIG. 1 is a morphological diagram of the colony of Lactobacillus harbinensis M1 of the present invention;

FIG. 2 is a morphological diagram of the thallus of Lactobacillus harbinensis M1 of the present invention.

### DETAILED DESCRIPTION

In order to better understand the present invention, the following further describes the present invention with reference to the accompanying drawings and embodiments, but the embodiments do not limit the protection scope of the present invention in any way.

In the following embodiments:

(1) For Gram staining, shape observation and common physiological and biochemical identification medium, please refer to “Classification, Identification and Test Methods of Lactic Acid Bacteria” (1999 edition) edited by Ling Daiwen.

(2) Improved MRS liquid medium (g/L) (for the cultivation of lactic acid bacteria):

Beef extract 10 g, peptone 10 g, yeast extract 5 g, glucose 20 g, Tween-80 1 mL, K₂HPO₄.3H₂O 2 g, NaAc.₃H₂O 5 g, triammonium citrate 2 g, MgSO₄.7H₂O 0.58 g, MnSO₄H₂O 0.25 g; 1 L of distilled water, pH 6.4±0.2 (MRS solid medium further includes 0.7-2% of agar on the basis of liquid medium), sterilized at 121° C. for 15 min.

(3) Analysis of Drug Resistance

According to the criteria for determining bacterial resistance (2012 edition) stipulated by the European Food Safety Agency, antibiotics including ampicillin, vancomycin, gentamicin, kanamycin, streptomycin, erythromycin, clindamycin, tetracycline and chloramphenicol were used to prepare antibacterial stock solutions with a concentration of 5120 μg/mL. They were diluted to the required concentration by a diluent preparation method before use, and were added to the corresponding medium according with certain proportion. 9.0 mL of the modified MRS agar media were added into large test tubes. After sterilizing at 121° C. for 15 min, they were placed in a water bath (50° C.) at a constant temperature for later use. 1.0 mL of the antibacterial diluents were pipetted into the large test tubes, followed by vortexing and mixing immediately. The diluents were then poured into a sterile petri dish, and bacteriostatic plates with a certain drug concentration (μg/mL) were prepared after solidification. For each antibiotics, 8 concentration gradient antibacterial plates were prepared according to a ½ dilution method. At the same time, a control plate without any drug was prepared. 1 μL (10⁹ CFU/mL) of bacterial suspensions were dipped and inoculated on the surface of the plates and incubated in 37° C. incubators for 16-20 hours. At the same time, a blank plate without inoculation was used as a control. The minimum inhibitory concentration (MIC) at which the bacteria does not grow on the plate was recorded. 3 parallel experiments were conducted. The lactic acid bacteria susceptibility result was shown in Table 1 determined according to the relevant standards:

TABLE 1

| | S. harbinensis M1 | L. casei-01 |
|---|---|---|
| | Turning point | Turning point |
| | of antibiotic | of antibiotic |
| | resistance | resistance |
| Antibiotics | (μg/mL) | (μg/mL) |
| ampicillin | 4 | 4 |
| vancomycin | No requirement | No requirement |
| gentamicin | 16 | 32 |
| kanamycin | 64 | 64 |
| streptomycin | 64 | 64 |
| erythromycin | 1 | 1 |
| clindamycin | 1 | 1 |
| tetracycline | 8 | 4 |
| chloramphenicol | 4 | 4 |

(4) Tolerance Analysis to Gastric Juice, Intestinal Juice and Bile Salt

Gastrointestinal juice tolerance: add 50 mL modified MRS liquid medium to a 100 mL Erlenmeyer flask, sterilize at 121° C. for 15 min, inoculate with lactic acid bacteria after cooling, and cultivate overnight at 37° C. to prepare a seed culture solution for later use. Simulated gastric juice: dilute 0.27 g of pepsin in 90 mL sterile phosphate buffer solution PBS, adjust to pH=3 with hydrochloric acid, filter with a 0.22 μm disposable syringe filter and sterilize for use. Simulated intestinal juice: dilute 0.1 g of trypsin in 100 mL sterile phosphate buffer solution PBS, adjust to pH=8.0 with sodium hydroxide, filter with a 0.22 μm disposable syringe and sterilize for later use. The cultured lactic acid bacteria were inoculated into the simulated gastric juice (pH=3) at an amount of 10⁹ CFU/mL, and samples were taken every hour from 0 h to 3 h to calculate the number of viable bacteria using a dilution coating method; after 3 hours, the bacteria were transferred to simulated intestinal juice (pH=8), samples were taken every hour from 0 h to 12 h to calculate the number of viable bacteria by a dilution coating method. 3 parallel experiments were conducted.

Simulated bile salt tolerance: add 0.3 g of porcine bile salt to 100 mL of modified MRS liquid medium, sterilize at 121° C. for 15 min, and cool to 37° C. The cultured lactic acid bacteria were inoculated in a MRS liquid medium containing 0.3% of porcine bile salt at an amount of 10⁹ CFU/mL, samples were taken every hour from 0 h to 11 h, and the absorbance at 620 nm was read. 3 parallel experiments were conducted. The calculation formula for the survival rate of bacteria is as follows: X (%)=*100%

(5) Test of Antibacterial Activity by an Oxford Cup Diffusion Method

The bacteria were activated with a MRS medium for 2-3 generations. The pathogenic bacteria were activated with LB medium for 2-3 generations for later use. The bacteria-containing MRS liquid medium was centrifuged at 10000 r/min at 4° C. for 5 min; the supernatant was divided into two aliquots, wherein one was left untreated, and the other was adjusted to pH 7.0. The supernatant was filtered through a 0.22 μm disposable syringe filter, and the antibacterial activity of the filtrate was tested by an Oxford cup diffusion method. A 1% pathogenic bacteria (Listeria monocytogenes, Salmonella typhimurium, Escherichia coli 0157, Enterobacter sakazakii, Streptococcus hemolyticus and Staphylococcus aureus) suspension (10⁸ CFU/mL) was poured into a sterilized dish. After solidification, it was placed on an agar plate by a sterilized Oxford cup. 60 μL of the filtrate was injected into the Oxford cup, and incubated at 37° C. for 24 hours. The size of the inhibition zone was recorded. 3 parallel experiments were conducted.

(6) Adhesion to Caco-2 Cells

The bacteria were inoculated in MRS medium and cultured at 37° C. for 24 hours. After centrifugation, they were collected, washed twice with a sterile solution, and finally re-suspended in a solution. The absorbance was measured at 600 nm, and the concentration of the bacteria was adjusted to 10⁹ CFU/mL (OD600=0.57) followed by centrifugation (6000 g, 5 min). The supernatant was removed, DMEM+10% bovine serum without Penicillin and Streptomycin was added with the same volume of the supernatant and mixed. Using DMEM+10% fetal bovine serum as a basic medium, Caco-2 cells were inoculated into cell culture plates, and cultured at 37° C. and 5% CO₂ for 10 days. When the degree of cell aggregation reached 90%-100%, the plates were washed three times with sterile PBS followed by adding 1 mL of the above lactic acid bacteria suspension (10⁹ CFU/mL) and incubating at 37° C., 5% CO₂ for 2 hours. Then, the plates were washed three times with sterile PBS, 1 mL of pancreatin was used for digestion, followed by sampling and calculating the number of viable bacteria by a dilution coating method. 3 parallel experiments were conducted.

(7) Utilization of Oligosaccharides

Lactic acid bacteria were respectively inoculated into MRS medium containing only glucose, sucrose, stachyose, or raffinose, cultured at 37° C. for 24 hours, and the absorbance at 600 nm was read. 3 parallel experiments were conducted. The formula for calculating the relative growth rate of lactic acid bacteria is as follows:

X (%)=*100%

(8) Analysis of Titratable Acidity of Fermented Soymilk

Soybeans that are not damaged and mildewed were mixed with water of 6 times the mass of the soybeans. Then 0.5% NaHCO₃ was added, followed by stirring and soaking at room temperature for 14 hours. 85° C. water was added, and hot refining was performed based on a soybean to water ratio of 1:8 (g:mL). Pure soy milk was obtained by filtering through a 180-mesh sieve, and was sterilized at 100° C. for 15 min. Single bacteria starter was added at an amount of 10⁶ CFU/mL, followed by fermentation in a constant temperature incubator at 37° C. for 24 h and post-mature at 4° C. for 24 h. Samples were taken and the number of viable bacteria was calculated by a dilution coating method. The acidity value was measured in accordance with GB 5413.34-2010 “Determination of Acidity of Milk and Dairy Products”: stir the sample with a glass rod, accurately weigh 10 g of the sample, and add 20 mL of CO₂-free distilled water; after mixing, add 0.5 mL of phenolphthalein indicator, titrate with 0.1 N NaOH standard solution until the solution is reddish and does not fade within 30 s, record the volume of sodium hydroxide consumed, and conduct 3 parallel experiments. The acidity was calculated according to the following formula:

X=(C×V×100)/(m×0.1)

X—acidity of the sample, unit: ° T;

C—concentration of sodium hydroxide standard solution, unit: mol/L;

V—volume of the sodium hydroxide standard solution consumed, unit: mL;

m—mass of the sample, unit: g.

(9) Analysis of volatile aromatic substances by solid phase microextraction-GC/MS (SPME-GC/MS): 50/30 μm DVB/CAR/PDMS extraction fiber head (SPME) was aged for 30 min at the inlet (270° C.). 5.0 g of fermented soybean milk was mixed with distilled water at a ratio of 1:1 and added to a 25 mL headspace bottle. The heating temperature was 40° C. After heating for 10 minutes, a SPME needle was inserted, and after 30 minutes of adsorption, the needle was inserted into an injection port of the GC/MS chromatograph for analysis. Gas chromatographic conditions: injection port temperature 300° C.; heating program: 35° C. for 2 minutes, heat to 110° C. at 5° C./min to, keep the temperature for 8 min, heat to 240° C. at 15° C./min, and keep the temperature for 5 min; split ratio 30:1; mass spectrometry conditions: mass spectrometer interface temperature 250° C.; ion source temperature 230° C.; quadrupole temperature 150° C.; ionization mode: EI; scan mode: full scan, mass range: 33˜400 m/z; Solvent delay: 0.1 min.

(10) Sensory and flavor evaluation: the score was based on a 9-point system, with 1 being the worst and 9 being the best. Thirty panelists were invited to rate the sample's odor, appearance, taste, texture and overall acceptability at 20° C.

(11) A positive control Lacticaseibacillus casei-01 was used, which was from Danish Kohansen Co., Ltd.

### Example 1: Screening and Identification of Strain

The first step was sampling and plate separation: use sterile sampling bottles to collect naturally fermented acidified yellow serofluid on site and bring them back at low temperature. Immediately dilute them to a concentration of 10⁻³, 10⁻⁴, and 10⁻⁵ by sterile water respectively, and spread on modified MRS solid media. Then put them in a constant temperature incubator at 37° C. for 48 hours. Pick out the suspected colonies and separate by a streak plate method, repeat 4 to 5 times until a pure single colony was obtained. Inoculate the purified single colony in MRS semi-solid medium by puncture inoculation and store in a refrigerator at 4° C.

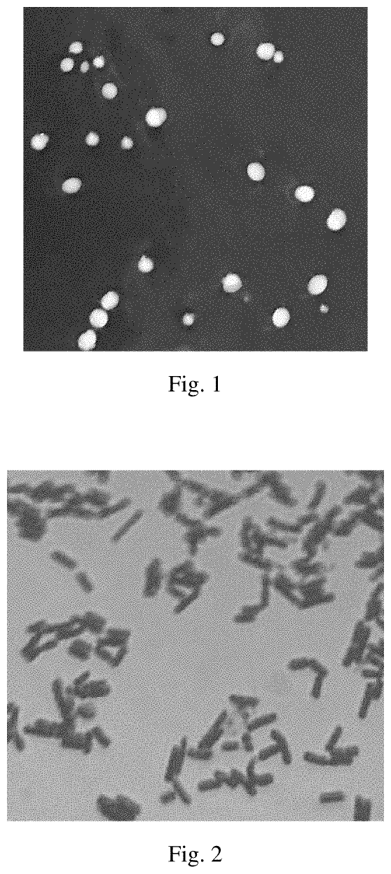

The second step was morphological observation and physiological and biochemical experiments: the colony of the strain on the MRS medium was round, off-white, translucent, wet and smooth (FIG. 1); the Gram stain and cell shape observation showed that thallus of the strain M1 was short rod-shaped and grew in pairs or piles (see FIG. 2). The results of routine physiological and biochemical experiments (see Table 2) showed that the strain M1 was a Gram-positive, catalase-negative, hetero-fermentative bactacin growing in the temperature range of 20-45° C. Table 2 shows the physiological and biochemical characteristics of the Schleiferilactobacillus harbinensis M1 strain of the present invention;

TABLE 2

| Item | M1 |
|---|---|
| Catalase | − |
| glucose | + |
| Starch hydrolysis test | + |
| Litmus Milk | + |
| Indole test | − |
| Gelatin Liquefaction Test | − |
| Acetyl methyl methanol | (+) |
| test (V-P) | |
| Hydrogen sulfide test | − |
| Exercise examination | − |
| Glucose fermentation | heterogeneous |
| Suitable growth | 20~45° C. |
| temperature | |

The third step was molecular identification: the obtained strain M1 was activated and cultivated, and then sent to a professional testing organization for sequencing, so as to obtain the 16S rDNA sequence (sequence as shown in SEQ.ID.NO.1 of the sequence listing). The sequence was compared in the NCBI gene bank to find standard strains KT897917.1 (Schleiferilactobacillus harbinensis strain LH-1), KF312693.1 (Schleiferilactobacillus harbinensis strain TCP001) and NR_113969.1 (Schleiferilactobacillus harbinensis strain NBRC100982) closely related to the strain. M1's 16S rDNA sequence was compared with that of the standard strains to conduct a similarity analysis, and the sequence homology between M1 and Schleiferilactobacillus harbinensis strain LH-1 was higher than 98% (see Table 3), indicating that they belong to the same species. The strain was identified as Schleiferilactobacillus harbinensis combined with the morphology of the colony and thallus, and the physiological and biochemical characteristics.

Table 3 shows the BLAST sequence comparison of the Schleiferilactobacillus harbinensis M1 strain of the present invention based on the 16S rDNA sequence.

TABLE 3

| | sequence | |
|---|---|---|
| Standard strains | homology | NCBI No. |
| Schleiferilactobacillus harbinensis | 99% | KT897917.1 |
| strain LH-1 | | |
| Schleiferilactobacillus harbinensis | 99% | KF312693.1 |
| strain TCP001 | | |
| Schleiferilactobacillus harbinensis | 99% | NR_113969.1 |
| strain NBRC100982 | | |

The strain was deposited in the Guangdong Microbial Culture Collection Center in 5^{th} Floor, Building 59, No. 100, Xianlie Middle Road, Guangzhou on Dec. 20, 2017, with a deposit number of GDMCC No. 60305.

### Example 2: Antibiotic Sensitivity

The first step was the preparation of lactic acid bacteria suspension: add 50 mL of modified MRS liquid medium to a 100 mL Erlenmeyer flask, sterilize at 121° C. for 15 min, inoculate Schleiferilactobacillus harbinensis M1 and Lacticaseibacillus casei-01 strains respectively, cultivate overnight at 37° C., and store in a refrigerator at 4° C. for later use.

The second step was the preparation of antibacterial plates: antibiotics including ampicillin, vancomycin, gentamicin, kanamycin, streptomycin, erythromycin, clindamycin, tetracycline and chloramphenicol were used to prepare antibacterial stock solutions at a concentration of 5120 m/mL. The solutions were diluted to the required concentration before use according to a diluent preparation method. 1.0 mL of the antibacterial diluents were pipetted into large test tubes with 9.0 mL of modified MRS agar media, followed by vortexing and mixing immediately. Then, they were poured on sterile plates. After solidification, antibacterial plates with a certain antibiotic concentration (m/mL) were obtained. At the same time, a control plate without antibiotic was prepared.

The third step was antibiotic sensitivity analysis: 14, of bacterial suspension (10⁹ CFU/mL) was inoculated on the surface of the plates and incubated in a 37° C. incubator for 16-20 hours. At the same time, a blank plate without inoculation was used as a control. The minimum inhibitory concentrations (MIC value) at which the strain did not grow on the plates were observed and recorded. The results showed that, like the positive control strain Lacticaseibacillus casei-01, the MIC values of Schleiferilactobacillus harbinensis M1 against the above nine antibiotics were all less than the turning point of antibiotic resistance (see Table 4), which met the EFSA safety standards.

TABLE 4
shows the results of the drug resistance analysis of the
Schleiferilactobacillus harbinensis M1 strain of the present invention;

| | S. harbinensis M1 | | L. casei-01 | |
|---|---|---|---|---|
| | Turning point | | Turning point | |
| | of antibiotic | MIC | of antibiotic | MIC |
| | resistance | value | resistance | value |
| Antibiotics | (μg/mL) | (μg/mL) | (μg/mL) | (μg/mL) |
| ampicillin | 4 | 1 | 4 | 1 |
| vancomycin | No requirement | 2 | No requirement | 4 |
| gentamicin | 16 | 16 | 32 | 16 |
| kanamycin | 64 | 16 | 64 | 16 |
| streptomycin | 64 | 8 | 64 | 16 |
| erythromycin | 1 | 0.5 | 1 | 0.5 |
| clindamycin | 1 | 0.5 | 1 | 0.125 |
| tetracycline | 8 | 2 | 4 | 2 |
| chloramphenicol | 4 | 2 | 4 | 4 |

### Example 3: Tolerance to Digestive Juices

The first step was the preparation of lactic acid bacteria suspension: add 50 mL modified MRS liquid medium to a 100 mL Erlenmeyer flask, sterilize at 121° C. for 15 minutes, inoculate Schleiferilactobacillus harbinensis M1 and Lacticaseibacillus casei-01 strains respectively, cultivate overnight at 37° C., and store in a refrigerator at 4° C. it for later use.

The second step studied the tolerance to simulated gastric juice and intestinal juice: inoculate the cultured lactic acid bacteria into simulated gastric juice (pH=3) at an amount of 10⁹ CFU/mL, and then transfer to simulated intestinal juice (pH=8) after 3 hours. Sampling was conducted every hour from 0 h-12 h to calculate the number of viable bacteria by a dilution coating method. The results showed that the remaining viable amount of Schleiferilactobacillus harbinensis M1 in simulated gastric juice and intestinal juice was 2.76 log CFU/mL, and the remaining viable amount of Lacticaseibacillus casei-01 was 2.98 log CFU/ml, showing that there was no significant difference between them.

The third step studied the tolerance to bile salts: inoculate the cultured lactic acid bacteria into MRS liquid medium containing 0.3% of porcine bile salts at an amount of 10⁹ CFU/mL, sample in every hour from 0 h to 11 h and read the absorbance at 620 nm to calculate the survival rate of bacteria. The results showed that the survival rate of Schleiferilactobacillus harbinensis M1 in the bile salt tolerance test was 94.3%, and the survival rate of the positive control Lacticaseibacillus casei-01 was 98.1%. Both of them have high tolerance to bile salts.

### Example 4: Inhibition Effect on Pathogenic Bacteria

The first step is to prepare cell-free supernatant of lactic acid bacteria: add 50 mL of modified MRS liquid medium in a 100 mL Erlenmeyer flask, sterilize at 121° C. for 15 minutes, inoculate Schleiferilactobacillus harbinensis M1 and Lacticaseibacillus casei-01 strains, and incubate at 37° C. for 24 hours. Centrifuge the above-prepared bacterial suspensions at 10000 r/min and 4° C. for 5 min. Divide the supernatants into two equal parts, wherein one part was left untreated and the other part was adjusted to pH 7.0. Filter the supernatants obtained with a 0.22 μm disposable syringe filter and store in a refrigerator at 4° C. for later use.

TABLE 5
shows the results of the antibacterial activity analysis of the Schleiferilactobacillus harbinensis
M1 strain of the present invention.

| | Pathogenic bacteria | | | | | | | | | | | |
|---|---|---|---|---|---|---|---|---|---|---|---|---|
| | Listeria | | Salmonella | | Escherichia | | Enterobacter | | Streptococcus | | Staphylococcus | |
| | monocytogenes | | typhimurium | | coli O157 | | sakazakii | | hemolyticus | | aureus | |
| Lactic | (mm) | | (mm) (mm) | | (mm) | | (mm) | | (mm) | | (mm) | |
| acid | No pH | pH = | No pH | pH = | No pH | pH = | No pH | pH = | No pH | pH = | No pH | pH = |
| bacteria | adjust | 7 | adjust | 7 | adjust | 7 | adjust | 7 | adjust | 7 | adjust | 7 |
| S. harbinensis | 8.2 | 7.0 | 8.8 | 10.7 | 8.2 | 7.0 | 10.7 | 12.3 | 7.0 | 7.0 | 8.5 | 8.5 |
| M1 | | | | | | | | | | | | |
| L. casei -01 | 11.7 | 7.0 | 7.7 | 7.0 | 7.5 | 7.0 | 11.5 | 7.0 | 7.0 | 7.0 | 11.5 | 7.0 |

The second step was antibacterial activity analysis: an Oxford cup diffusion method was used to test the cell-free supernatant of lactic acid bacteria against pathogenic bacteria including Listeria monocytogenes, Salmonella typhimurium, Escherichia coli 0157, Enterobacter sakazakii, Streptococcus hemolyticus and Staphylococcus aureus. The results show that compared with the positive control strain, the supernatant of Schleiferilactobacillus harbinensis M1 has a stronger ability to inhibit the tested pathogenic bacteria, and may have produced bacteriocin antibacterial substances (Table 5).

### Example 5: Adhesion to Small Intestinal Epithelial Cells Caco-2

The first step was to prepare a suspension of lactic acid bacteria: add 50 mL of modified MRS liquid medium to a 100 mL Erlenmeyer flask, sterilize at 121° C. for 15 min, inoculate Schleiferilactobacillus harbinensis M1 and Lacticaseibacillus casei-01 strains respectively, incubate at 37° C. for 24 hours, and adjust the cell concentration to 10⁹ CFU/mL by DMEM+10% bovine serum without Penicillin and Streptomycin.

The second step was cell adhesion experiment: inoculate Caco-2 cells into a cell culture plate and culture for 10 days at 37° C. and 5% CO₂. Wash three times with sterile PBS, add 1 mL of the above lactic acid bacteria suspension (10⁹ CFU/mL), continue to incubate for 2 hours at 37° C. and 5% CO₂, again wash three times with sterile PBS, and digest with 1 mL of pancreatin. Sample was taken to calculate the number of viable bacteria by a dilution coating method. The results showed that the number of adhered bacteria of Schleiferilactobacillus harbinensis M1 to the small intestinal epithelial cell Caco-2 was 5.21 log CFU/mL, while the number of adhered bacteria of the positive control Lacticaseibacillus casei-01 was 5.22 log CFU/mL, indicating that both of them had good adhesion to the small intestinal epithelium.

### Example 6: Utilization characteristics of oligosaccharides

The first step was the preparation of lactic acid bacteria suspension: add 50 mL modified MRS liquid medium to a 100 mL Erlenmeyer flask, sterilize at 121° C. for 15 min, inoculate Schleiferilactobacillus harbinensis M1 and Lacticaseibacillus casei-01 strains respectively, cultivate overnight at 37° C., and store in a refrigerator at 4° C. for later use.

The second step was the utilization of oligosaccharides: inoculate the activated strains into MRS medium containing only glucose, sucrose, stachyose, or raffinose, incubate at 37° C. for 24 hours, and read the absorbance at 600 nm. The results showed that the relative growth rates of Schleiferilactobacillus harbinensis M1 in sucrose, stachyose and raffinose were 121.59%, 94.20%, and 103.08%, respectively, while the relative growth rates of the positive control strain Lacticaseibacillus casei-01 in sucrose, stachyose and raffinose were 98.27%, 48.25%, 91.91%, respectively, indicating that the strain has high utilization rates of sucrose, stachyose and raffinose substantially the same as that of glucose, and is suitable for growth in legume substrates or plant raw materials.

### Example 7: Number of Viable Bacteria and Volatile Components of Fermented Soymilk

The first step was the preparation of fermented soymilk and the analysis of viable cell amount: divide the pure soymilk filtered through a 180-mesh sieve into 10 ml large test tubes and sterilize at 100° C. for 15 min. Add a single strain at an amount of 10⁶ CFU/mL, ferment in a constant temperature incubator at 37° C. for 24 hours, and then place in a refrigerator at 4° C. for 24 hours for post-mature. The number of viable bacteria and titratable acidity in the sample were analyzed. The results showed that the Schleiferilactobacillus harbinensis M1 fermented soymilk had a number of viable bacteria of 9.08 log CFU mL⁻¹ and an acidity value of 44.35° T, while the positive control Lacticaseibacillus casei-01 fermented soymilk had a number of viable bacteria of 8.42 log CFU mL⁻¹ and an acidity value of 36.91° T. There was significant difference between the two, indicating that Lactobacillus harbinensis M1 is more suitable for fermentation in soy milk.

The second step was to analyze the volatile components of fermented soymilk: solid-phase microextraction-gas-mass spectrometry (SPME-GC/MS) was used to analyze the volatile aromatic substances of fermented soymilk. The results are shown in Table 6. From the results in the table, (1) soymilk fermented by Schleiferilactobacillus harbinensis M1 can significantly reduce the content of beany flavor substances such as n-hexanal (100%), nonanal (100%), 1-octen-3-ol (31%), etc. However, soymilk fermented by the control strain Lacticaseibacillus casei-01 had a significantly weaker ability to reduce beany flavor substances such as n-hexanal (61%) and 1-octene-3-ol (13%), and even increased some of the beany flavor components such as n-hexanol and 2-ethylfuran. (2) Soymilk fermented by Schleiferilactobacillus harbinensis M1 strain can produce rich aroma components, wherein the content of diacetyl and acetoin, which are the characteristic flavor components of milk, were 7 times and 202 times of that of the soymilk fermented by control Lacticaseibacillus casei-01, respectively, leading to a unique milk flavor of the product. There was no report on producing milk flavor volatile substances by Schleiferilactobacillus harbinensis.

Table 6 shows the effects of the Lactobacillus harbinensis M1 strain of the present invention on the volatile components of fermented soymilk.

TABLE 6

| | Soymilk sample | | |
|---|---|---|---|
| | | S. harbinensis M1 | L. casei-01 |
| Volatile aroma | | fermented | fermented |
| component | soymilk | soymilk | soy milk |
| Main component of | | | |
| beany flavor | | | |
| Hexanal | 74.90 | N.D. | 29.18 |
| Nonanal | 8.04 | N.D. | N.D. |
| Benzaldehyde | 9.41 | 8.46 | 8.30 |
| (E, E)-2,4-Decadienal | 4.69 | 3.77 | 3.30 |
| 3-Octanone | 24.15 | 17.48 | 23.08 |
| 3-Octanol | 22.52 | 16.40 | 19.41 |
| 1-Octen-3-ol | 128.13 | 87.44 | 111.40 |
| 1-Hexanol | 50.17 | 33.18 | 53.70 |
| 2-Ethyl furan | 13.36 | 11.42 | 14.68 |
| 2-Pentyl furan | 38.85 | 46.81 | 53.87 |
| Heptanoic acid | N.D. | 0.37 | 3.33 |
| Main component | | | |
| of aroma | | | |
| Dodecanal | N.D. | 0.43 | N.D. |
| (E)-2-Octenal | N.D. | 2.97 | 0.88 |
| (E)-2-Nonenal | N.D. | 1.45 | 0.53 |
| 2,3-Butanedione | N.D. | 74.85 | 0.37 |
| 2-Octanone | N.D. | 0.42 | 0.43 |
| Acetoin | N.D. | 29.13 | 4.16 |
| 2-Nonanone | N.D. | 1.33 | N.D. |
| Acetic acid | N.D. | 62.72 | 47.23 |
| Hexanoic acid | N.D. | 3.20 | 1.48 |

The third step was sensory evaluation: sensory evaluation tests were carried out on the two fermented soymilk. The results showed that the sensory evaluation scores of Schleiferilactobacillus harbinensis M1 fermented soymilk were: overall acceptability 8.13, smell 8.38, appearance 7.38, taste 8.63 and texture 8.13; while the sensory evaluation scores of Lacticaseibacillus casei-01 fermented soymilk were: overall acceptability 6.5, smell 6.00, appearance 6.13, taste 6.25 and texture 6.75. The scores of Schleiferilactobacillus harbinensis M1 fermented soymilk were significantly higher than those of the Lacticaseibacillus casei-01 fermented soymilk.

It can be seen from the above examples that the strain Schleiferilactobacillus harbinensis M1 isolated from acidified yellow serofluid of bean curd has the characteristics of wide growth temperature range and easy cultivation (Example 2). Like Lacticaseibacillus casei-01, it has good tolerance to digestive juices (Example 3), adhesion to small intestinal epithelial cells (Example 4), and inhibition on pathogenic bacteria (Example 5). Especially the strain has a very good inhibitory effect on pathogenic bacteria, and may produce bacteriocin antibacterial substances. At the same time, the strain has a high utilization rate of oligosaccharides, and can grow in a matrix rich in oligosaccharides such as sucrose, stachyose, raffinose, etc. It is also an ideal starter for fermenting plant-based foods such as soymilk (Example 6). The fermented soymilk prepared by the strain has higher viable cell count and acidity; the strain can significantly reduce the beany flavor components in the soymilk and increase unique milk aroma components, significantly improving the flavor and taste of soymilk (Example 7) and having a good application prospect.

The Schleiferilactobacillus harbinensis of the present invention is a Gram-positive glucose hetero-fermentative bactacin, which has a wide growth temperature range (grow normally at 20-45° C.) and has a fast growth speed. The results of conventional physiological and biochemical experiments show that the strain M1 is Gram-positive, catalase negative, and non-exercise. The 16S rDNA sequence was compared by BLAST to find standard strains KT897917.1 (Schleiferilactobacillus harbinensis strain LH-1), KF312693.1 (Schleiferilactobacillus harbinensis strain TCP001) and NR_113969.1 (Schleiferilactobacillus harbinensis strain NBRC100982) having close relationship with the strain. The 16S rDNA of the M1 strain was analyzed for similarity with the standard bacteria, and it was identified as Schleiferilactobacillus harbinensis.

The strain is sensitive to antibiotics ampicillin, vancomycin, gentamicin, kanamycin, streptomycin, erythromycin, clindamycin, tetracycline and chloramphenicol, the safety of which meets the requirements of EFSA.

The strain's tolerance to simulated gastric juice (pH=3, 3 h), intestinal juice (pH=8, 12 h) and bile salts, and its ability to adhere to small intestinal epithelial cells Caco-2 are all similar to those of the control strain Lacticaseibacillus casei-01, indicating that it can be colonized on the epithelial cells of the small intestine and exhibit probiotic effects.

The supernatant of this strain has a significant inhibitory effect on pathogenic bacteria such as Listeria monocytogenes, Salmonella typhimurium, Escherichia coli 0157, Enterobacter sakazakii, Streptococcus hemolyticus and Staphylococcus aureus. After adjusting the pH of the supernatant to 7.0, it still has a strong antibacterial ability, indicating that the strain may produce bacteriocin antibacterial substances.

The utilization rates of the strain of sucrose, stachyose and raffinose are significantly higher than that of the control bacteria, and the fermented soymilk prepared by the strain has a higher number of viable bacteria and higher acidity value. In addition, the strain can also produce rich aroma components during fermentation of soymilk, wherein the content of diacetyl and acetoin, which are the characteristic aroma components of milk, are significantly higher than that of the soymilk fermented by the control bacteria. Also, the contents of beany flavor components are significantly reduced, wherein n-hexanal disappears completely. The sensory evaluation results show that the strain can significantly improve the flavor and taste of fermented soymilk, which may serve as an ideal starter for fermented soymilk and other plant raw materials, and have a broad application prospects.

The present invention is not restricted by the above-mentioned embodiments. Any other changes, modifications, substitutions, combinations, and simplifications made without departing from the spirit and principle of the present invention should be equivalent alternatives and are all included in the protection range of the present invention.

1. Schleiferilactobacillus harbinensis M1, characterized in that: the preservation number of the bacteria is GDMCC No. 60305.

2. Use of the Schleiferilactobacillus harbinensis M1 of claim 1 as probiotics.

3. The use of the Schleiferilactobacillus harbinensis M1 as probiotics according to claim 2, characterized in that, the Schleiferilactobacillus harbinensis M1 is sensitive to antibiotics including ampicillin, vancomycin, gentamicin, kanamycin, streptomycin, erythromycin. clindamycin, tetracycline and chloramphenicol, and the safety meets the requirements of EFSA.

4. The use of the Schleiferilactobacillus harbinensis M1 as probiotics according to claim 2, characterized in that, the supernatant of the Schleiferilactobacillus harbinensis M1 is effective on inhibitory of Listeria monocytogenes, Salmonella typhimurium, Escherichia coli 0157, Enterobacter sakazakii, Streptococcus hemolyticus and Staphylococcus aureus.

5. The use of the Schleiferilactobacillus harbinensis M1 as probiotics according to claim 2, characterized in that the Schleiferilactobacillus harbinensis M1 is resistant to gastric juice, intestinal juice and bile salts, and can be colonized on small intestinal epithelial cells.

6. The use of the Schleiferilactobacillus harbinensis M1 of claim 1 in fermented soybean milk.

7. The use of the Schleiferilactobacillus harbinensis M1 in fermented soybean milk according to claim 6, characterized in that the Schleiferilactobacillus harbinensis M1 has utilization rates of sucrose, stachyose and raffinose substantially the same as the utilization rate of glucose.

8. The use of the Schleiferilactobacillus harbinensis M1 in fermented soymilk according to claim 6, characterized in that the Schleiferilactobacillus harbinensis M1 completely removes the beany flavor component n-hexanal in the soymilk.

9. The use of the Schleiferilactobacillus harbinensis M1 in fermented soymilk according to claim 6, characterized in that the Schleiferilactobacillus harbinensis M1 increases the milk aroma components diacetyl and acetoin.


SEQUENCE LISTING (1 sequence)

SEQ ID NO: 1 - DNA, 1479 bp, Schleiferilactobacillus harbinensis M1
tggcaaggggggcggctataatgcaagtcgaacgaggtttggtcagtttgcggtggtgcttgcatcaccaattaccgatcaaaccgagtggcggacgggtgagtaacacgtgggtaacctgcccttcagcaggggataacatttggaaacagatgctaataccgtataaccacggagaccgcatggtctccgggtaaaagatggcgcaagctatcactgaaggatggacccgcggcgtattagccagttggtggggtaacggcctaccaaagcgatgatacgtagccgacctgagagggtaatcggccacattgggactgagacacggcccaaactcctacgggaggcagcagtagggaatcttccacaatggacgcaagtctgatggagcaacgccgcgtgagtgaagaaggctttcgggtcgtaaaactctgttattgaagaagaacgtgtgtgacagtaactggtcatgcagtgacggtattcaatcagaaagtcacggctaactacgtgccagcagccgcggtaatacgtaggtggcaagcgttgtccggatttattgggcgtaaagcgagtgcaggcggtcttttaagtctgatgtgaaagccttcggcttaaccgaagaagggcatcggaaactgggagacttgagtgcagaagaggagagtggaactccatgtgtagcggtgaaatgcgtagatatatggaagaacaccagtggcgaaggcggctctctggtctgtaactgacgctgaggctcgaaagcgtgggtagcaaacaggattagataccctggtagtccacgccgtaaacgatgaatactaagtgttggagggtttccgcccttcagtgctgcagctaacgcattaagtattccgcctggggagtacgaccgcaaggttgaaactcaaaggaattgacgggggcccgcacaagcggtggagcatgtggtttaattcgaagcaacgcgaagaaccttaccaggtcttgacatcttctgccaggctgagagatcagctgttcccttcggggacagaatgacaggtggtgcatggttgtcgtcagctcgtgtcgtgagatgttgggttaagtcccgcaacgagcgcaacccttatgatcagttgccagcattcagttgggcactctggtcagactgccggtgacaaaccggaggaaggcggggatgacgtcaaatcatcatgccccttatgacctgggctacacacgtgctacaatgggtggtacaacgagcagcgagaccgcgaggtcaagcgaatctctaaaaaccatcctcagttcggattgcaggctgcaactcgcctgcatgaagctggaatcgctagtaatcgcggatcagcacgccgcggtgaatacgttcccgggccttgtacacaccgcccgtcacaccatgagagtttgtaacacccaaagccggtgagacaaccgcaaggagtcagccgtctaagtgacaaaagccca